(19) United States
(12) Patent Application Publication
(10) Pub. No.: US 2003/0139690 A1
(43) Pub. Date: Jul. 24, 2003

(54) Device for in vivo measurement of pressures and pressure variations in or on bones

(76) Inventors: Nikolaus Aebli, Nz-Dunedin (NZ); Albert Graf, Morigen (CH); Peter H. Wunderli, Sutz (CH)

Correspondence Address: OSTROLENK FABER GERB & SOFFEN, 1180 AVENUE OF THE AMERICAS, NEW YORK, NY, 100368403

(21) Appl. No.: 10/169,875

(22) PCT Filed: Dec. 19, 2000
(86) PCT No.: PCT/CH00/00671

(30) Foreign Application Priority Data
Jan. 7, 2000 (EP) 00810012.5

(51) Int. Cl. A61B 5/103

(52) U.S. Cl. 600/587

(57) ABSTRACT

The device for the in vivo measurement of pressures and pressure variations in or on the human or animal body, more particularly inside or outside bones, is composed of an implantable probe (1) and of an evaluating unit (3), the probe (1) comprising an oscillating circuit (L-C) including a capacitor in the form of a pressure sensor (8, 30) and a coil (13), and the evaluating unit (3) comprising a resonance oscillating circuit capable of detecting the natural frequency (Fₑ) of the oscillating circuit, is variable according to variations of a dimension of the capacitor caused by pressure variations. The pressure sensor (8) is spatially separated from the coil (13) and connected to the latter by an electric conductor (15). On one hand, this allows small dimensions of the implantable pressure sensor, and on the other hand, a clear signal transmitted over a relatively long distance. In the first place, such a measuring device allows to monitor the accretion of a bone to a prosthesis, or quite generally, to detect the behaviour of a bone or another organ e.g. under load.

The present invention refers to a device for the in vivo measurement of pressures and pressure variations in or on bones or on dental implants according to the preamble of claim 1, one embodiment of the invention being particularly intended for monitoring the behaviour of prostheses.

There are different known devices and methods for measuring various parameters in vivo, for example from U.S. Pat. No. 4,281,667, disclosing a method and device for measuring the intracranial differential pressure where a sensor comprising two membranes is implanted in the head and the excursions of the membrane caused by the pressure difference are transmitted by an L-C oscillator to a receiver outside the body wherein the variations are recorded and available at any time. The device required therefor is relatively complicated due to the fact that two membranes are necessary and the oscillations of the membranes influence an electric oscillating circuit whose signals are subsequently transmitted, and the possible transmission distance is very short.

German Patent Application No. DE-A-38 41 429 also discloses a device for measuring the intracranial pressure where the pressure sensor comprises a terminating membrane whose oscillations act upon an oscillating circuit which measures the variations. The housing of the pressure sensor comprises a circuit board one side of which forms a capacitor with the membrane while a coil is disposed on the other side. This device involves important operative damages since a hole of 11-12 mm has to be drilled which also causes problems at the removal of the pressure sensor since bone and scar tissue will have covered the pressure sensor in the meantime.

European Patent No. EP-B-579 673 discloses a device for the inspection of implants where a component is fastened to a tooth implant and its natural frequency is detected by an appliance. The component is excited by a signal and examined for mechanical resonance.

Also, various test arrangements are known which allow to detect a loosened prosthesis by means of a vibration analysis where e.g. two connections are established in order to produce vibrations in the bone, on one hand, and to measure the resulting vibrations and to transmit them to an evaluating device, on the other hand.

On the background of this prior art, it is an object of the present invention to provide a device for the measurement of pressures and pressure variations in or on bones or on dental replacements, whose probe has a simple structure and is easy to implant and to remove, on one hand, and which is capable of transmitting signals over such a distance that the latter can be correctly recorded by a reading device outside the human or animal body, on the other hand. Another object of the invention is to provide such a design of the probe and of the reading device that it is suitable for various tasks inside the human or animal body. These objects are attained by the device as described in claim 1. Further advantages and embodiments are defined in the dependent claims.

The invention is explained in more detail hereinafter with reference to drawings of exemplary embodiments.

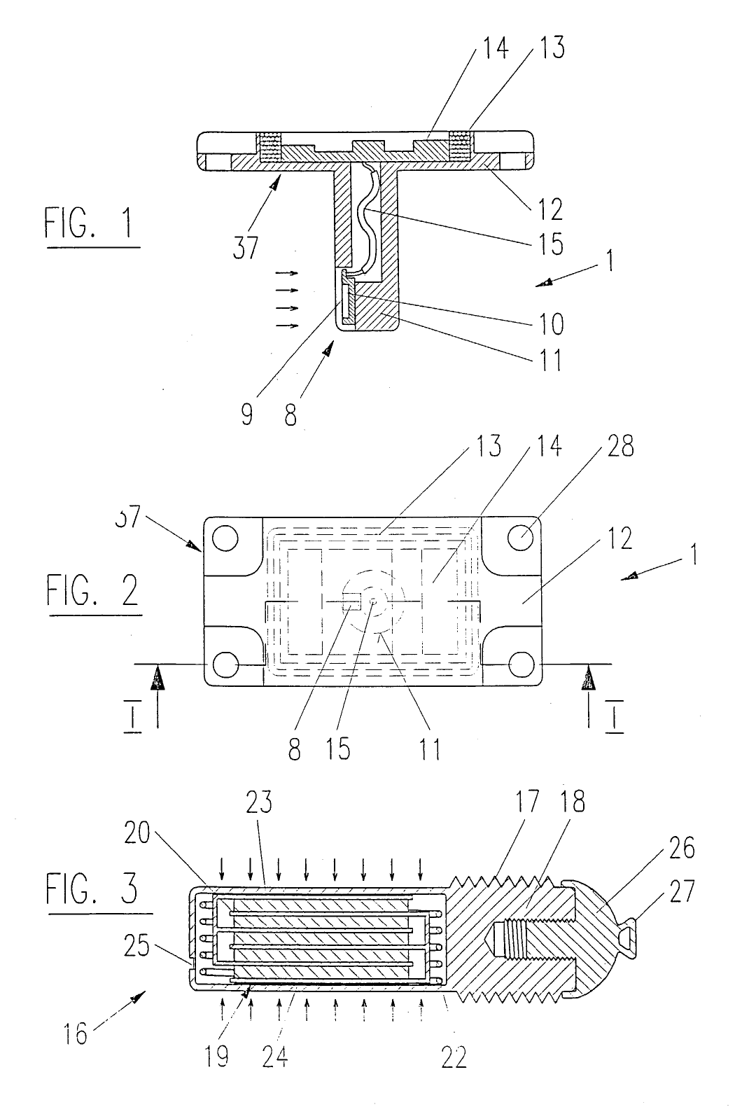

FIG. 1 shows a cross-section according to line I-I in FIG. 2 of a first exemplary embodiment of a probe according to the invention;

FIG. 2 shows a top view of the probe of FIG. 1;

FIG. 3 shows a second exemplary embodiment of a probe according to the invention;

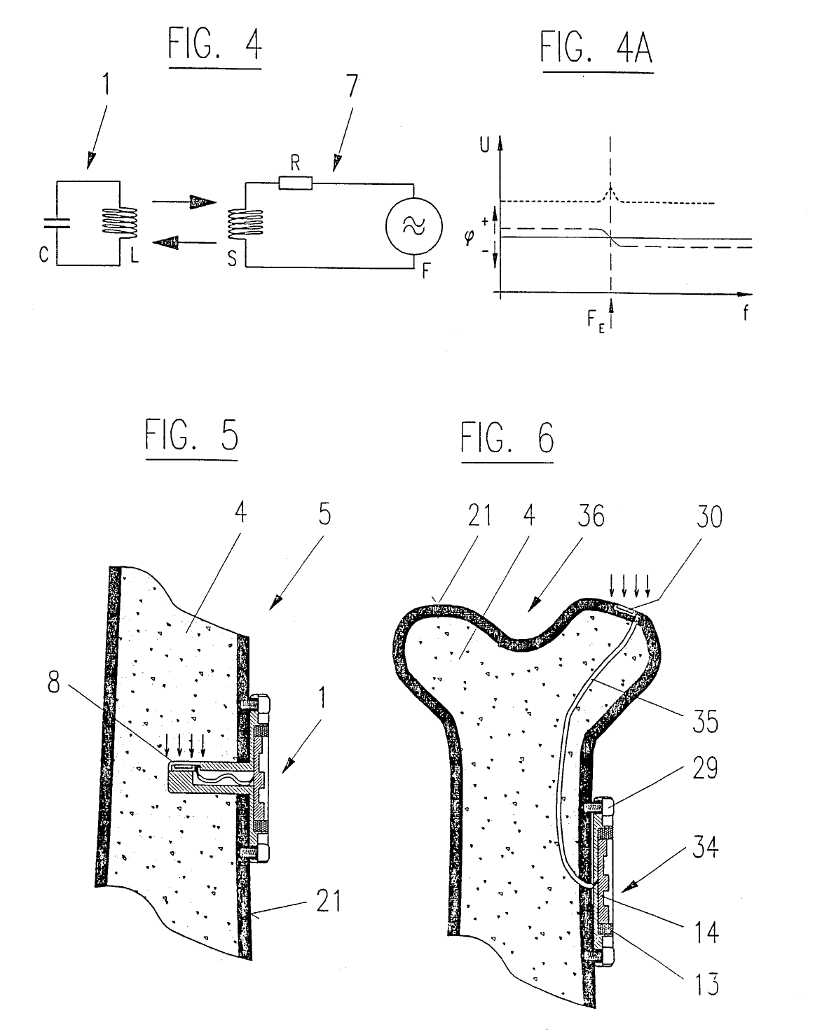

FIGS. 4, 4A schematically show the link between the probe and the receiver of the device;

FIG. 5 shows the probe of FIG. 1 implanted in a bone;

FIG. 6 shows an alternative embodiment of the probe of FIG. 5 on an articulation;

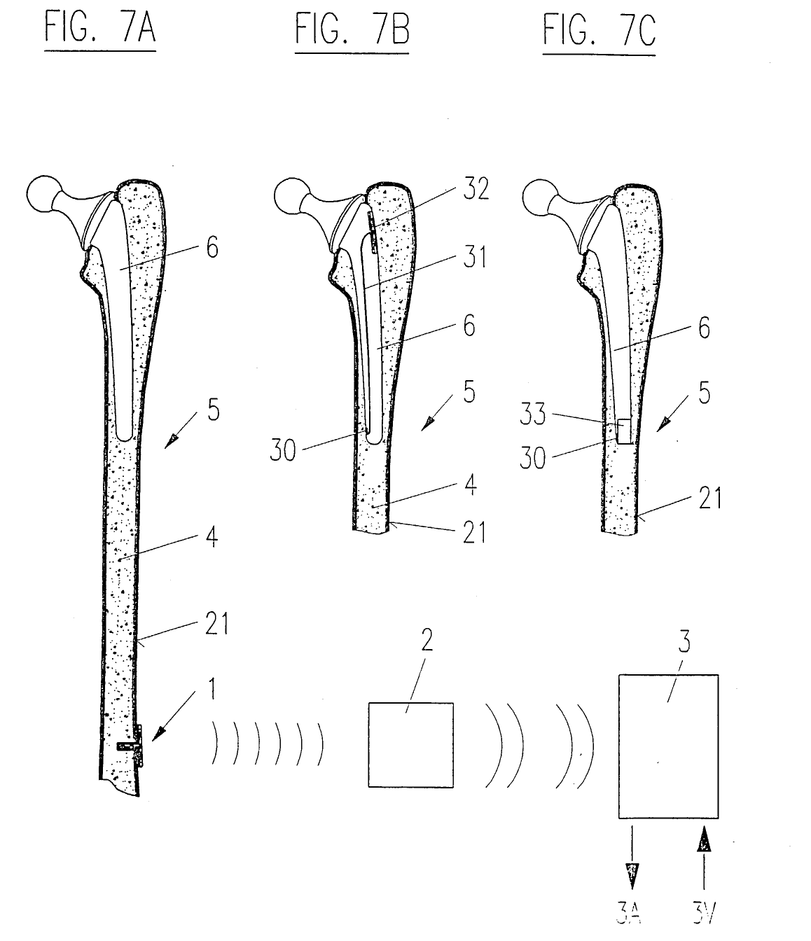

FIG. 7A schematically shows the probe of FIG. 1 in the medulla of a femur provided with a hip joint prosthesis;

FIG. 7B schematically shows an alternative embodiment of the probe of the invention on a hip joint prosthesis implanted in the femur; and

FIG. 7C shows an alternative embodiment of FIG. 7B.

Naturally, a probe intended for implantation should be as small as possible, but at the same time efficient enough to be able to send signals from inside the body to the surface of the body at least. Such a probe is schematically illustrated in FIG. 4, and an exemplary embodiment thereof is shown in FIG. 1. The probe forms an oscillating circuit comprising a capacitor C and a coil L which determine the natural frequency of the oscillating circuit. In this embodiment, coil L is invariable, whereas the membrane of capacitor C is more or less deformed by the pressure outside the probe, thus varying the capacity of the capacitor and therefore also the natural frequency of the oscillating circuit.

The natural frequency is detected and recorded externally, i.e. by means of a receiver outside the body comprising a resonance circuit, thus providing a reliable measuring value of the momentary pressure at the corresponding location.

A first embodiment of the probe is described with reference to FIGS. 1 and 2. Probe 1 has a T-shaped cross-section, the stem 11 of housing 37 comprising capacitive pressure sensor 8 and the crosspiece 12 comprising coil 13 and electronic circuit 14, which are connected to each other by an electric conductor 15 extending in the housing as well. The crosspiece further comprises bores 28 for bone screws 29 in order to fasten the probe to the bone.

Pressure sensor 8 shown in FIG. 1 consists of a capacitor comprising a membrane 9, which may be a silicon membrane, for example, and a variation of the distance between the membrane and capacitor surface 10 will result in a variation of the capacity and thus of the natural frequency of the oscillating circuit.

Alternatively, in the example according to FIG. 3, probe 16 may be directly screwed into a bone, more particularly a maxillary, for which purpose it comprises a pin 18 provided with a suitable threaded portion 17 allowing the probe to be screwed into the cortex 21 of the bone. In this embodiment, capacitor 19 is composed of capacitor plates 20 around which a coil 22 is wound. The two outer capacitor plates 23 and 24 are acting as membranes and are fastened to the inside of the housing surfaces, so that a variation of the height of the housing results in a variation of the thickness of the capacitor and thus of its capacity. Advantageously, the housing comprises two thinner portions 25 in order to be more compressible in the direction of its height. If this probe is fastened in the medulla, a cap 26 comprising a fastening loop 27 can be screwed onto threaded pin 18, or a dental replacement resp. a crown can be screwed on in the area of the cap.

In a further embodiment, not shown in the drawings, the membrane, for example made of titanium, is located at the end of the stem, which also can be made of titanium, and pretensioned by the uppermost of several movable parts of a capacitor and acting with reference to the static parts of the capacitor fixed within the stem. The movable parts are guided by a central pin being under the pressure of a spring. A movement of the membrane variates the capacity of the capacitor, which is transmitted like in the first embodiment.

The probes shown in FIGS. 1 to 3 and described may be used in this form or in modified forms in a device for the in vivo measurement of pressures and pressure variations. The device of the invention is schematically illustrated in FIG. 7 and is composed of the probe, the so-called interface 2, and of evaluating device 3.

Furthermore, FIGS. 4 and 4A schematically illustrate the operation of the measuring device. FIG. 4 only shows the schematic structure of the transmitter/receiver section 7 of the evaluating device, which comprises a frequency generator F, a power supply 3V, an output 3A, see FIG. 7, and a transmitter/receiver coil S which drives the oscillating circuit L-C of the probe and allows to detect the natural frequency Fₑ of the latter in the case of resonance, see FIG. 4A. In the present embodiment, the natural frequency may be in the vicinity of 1450 kHz, and the deviation of the frequency caused by a force of 1 N amounts to approx. 25 kHz. To those skilled in the art, it is apparent that different dimensions of the capacitor and of the coil and different forces will produce different natural frequencies. This measuring arrangement also allows to measure the static pressure, i.e. without a variation of the capacitor dimensions. The above indications shall be regarded as an example.

Such an arrangement further allows an evaluation both of the phase Φ and of the voltage V of the oscillating circuit. In an alternative embodiment, the receiver itself may deliver a D.C. voltage corresponding to the measured pressure, which is subsequently evaluated by means of existing apparatus such as computers, chart recorders, etc.

In view of the increasing tendency towards digital electronic circuits, it is understood that digital signals may be used instead of processing analog signals, thus allowing for an improved measuring accuracy and mainly for an increased insensitivity to interference and longer operating distances.

If the probe and the receiver are used alone, problems may arise due to the fact that the evaluating unit cannot always be positioned close enough to the probe. Therefore, in an advantageous embodiment of the invention according to FIG. 7, a so-called interface 2 is provided which receives the signals of the probe and allows their transmission over a greater distance of e.g. 20 to 100 m. To this end, the interface comprises a power supply e.g. in the form of a long-life battery or of solar cells or the like. The interface may either be operated directly on the body or under the skin in the vicinity of the probe. If the interface is implanted as well, it is obvious that it should be as small as possible and that in this case, according to present knowledge, the power supply should be a long-life battery. The interface is preferably flexible and capable of being externally attached to the skin at the location of the implant by means of a bandage while the measurements are being performed.

Alternatively, the interface may be divided into two component parts which are connected to each other by a cable. In this case, one portion consists of the antenna section, which is e.g. taped to the patient's skin and may be 1-2 mm thick and flexible, while the other portion contains the remaining electronics and the battery. In this manner, a single interface allows to transmit measuring values from a plurality of probes. If it is impossible, for example, to attach an interface to the neck or to another portion of the body because it is too large and uncomfortable, an antenna may be used in these places while the other portion of the interface is affixed to another portion of the patient's body or to the bed.

In the exemplary application according to FIG. 5 or 7A, probe 1 of FIG. 1 is fastened to resp. in a bone. In this case, pressure sensor 8 is positioned inside the medulla 4 of a femur 5 in which the shaft 6 of an artificial hip joint is implanted. Probe 1 is fastened to the bone by means of screws 29, and both coil 13 and electronic circuit 14 are located outside the bone. This solution offers the advantage of allowing a better transmission of the electric values over a greater distance as well as a substantially reduced size of the implantation hole in the bone.

This solution is based upon the realization that the space inside the bone, i.e. the medulla, may be considered as a communicating vessel, such that pressure variations at any point of the medulla will propagate in the latter and compensate each other. Consequently, the same pressure will be measured at any point in the medulla of a bone. Thus, in the present case according to FIG. 7, after the insertion of the prosthesis shaft 6 in the femur 5, the forces acting upon the prosthesis while standing or walking will be transmitted to the medulla and cause an increase of the pressure in the latter. Now, the better the prosthesis is anchored in the bone, the smaller the pressure increase, thereby providing an almost natural, i.e. physiological transformation and transmission of the forces. In other words, the extent of the pressure increase in the medulla is a measure of the quality of the anchorage of a prosthesis, which in turn allows a judgement of the osteo-integration, i.e. of the growth of the bone to the prosthesis or the cement mantle.

The recording of the pressure variations in the medulla allows to monitor the healing progress after the placement of a prosthesis, to determine the maximum allowable load on the prosthesis during convalescence, and to detect the occurrence of complications, especially of a loosening of the prosthesis, at an early stage.

However, the measuring device may also be used for the observation of operations on articulations since pressure variations may be indicative of the course of an operation.

In the embodiment according to FIG. 7B, the probe is divided, i.e. pressure sensor 30 is located at some point of the prosthesis shaft 6 and connected to coil 13 and electronic circuit 14, which are contained in a separate housing, by a longer conductor 31.

In the embodiment according to FIG. 7C, the entire probe 33 is located at the end of the prosthesis shaft.

Considering the example of FIG. 7, it is apparent that a probe may also be implanted in the medulla of other bones, for example at the articulations of the knee, of the shoulder, or of the elbow, and of course also in animals. Furthermore, if the probe is suitably dimensioned, the latter could e.g. be implanted in the oral cavity, i.e. in a tooth or in the jaw bone, in order to monitor the accretion of the bone to a dental implant, for example.

In FIG. 6, the probe is divided as in FIG. 7B, i.e. pressure sensor 30 is connected by an electric conductor 35 to an housing 34 containing coil 13 and electronic circuit 14, the pressure sensor being located at the tibia 36 of a knee joint, and housing 34 being fastened to the bone by means of screws 29.

Furthermore, a probe of this kind also allows to monitor other sequences of motions on human or animal skeletons which may produce a pressure or a pressure variation in the probe, more particularly if the probe is fastened in joints, on the bone, in muscles, tendons or various vessels.

Thus, the probe may be used in veterinary medicine, for example in order to perform a diagnosis of lameness in horses, for training surveillance, or for the classification of suitable soils with regard to stress of the articulations and the development of arthrosis.

In the examples, the probe is illustrated as being rectangular resp. in the form of a cube, but other, especially rounded or spherical shapes are possible as well, particularly if the probe is to be integrated in prostheses, implants, or joints.

In a further development of the invention, a plurality of probes may be used in the same body, the natural frequency of each probe being adjusted such that they operate on adjoining frequency bands. In this case, the receiver resp. the evaluating unit must be designed accordingly.

As already mentioned in the introduction, a probe of the invention may also be used for various other measurements. Thus, the recording of the pressure variations vs. time may be indicative of the development of arthroses. Furthermore, with a single probe or a plurality thereof, it is possible to measure specific pressure loads in joints resp. in parts of joints.

Thus, at least three different applications of the probe can be distinguished, namely:

a) intraossar, as previously described;

b) intraarticular, the probe being fastened between the parts of joints to be measured; and

c) extraossar, the probe being fastened on the outside of bones or in tendons, muscles, or various vessels.

The receiver may also be in the form of a miniaturized multichannel recorder capable of recording, in the receiver itself, the force variations of different probes over prolonged periods of time for ulterior evaluation while each probe can be identified from the outside.

1. Device for the in vivo measurement of pressures and pressure variations in or on a body, more particularly inside or outside bones or on dental implants, the device comprising an implantable probe and an evaluating unit, characterized in that the probe (1; 33) comprises an oscillating circuit (L-C) including a pressure sensor (8, 30) and a coil (13), and the evaluating unit (3) comprises a resonance oscillating circuit (7) capable of detecting the natural frequency (Fₑ) of the oscillating circuit, the natural frequency (Fₑ) of the oscillating circuit (L-C) being determined by the momentary pressure acting on the pressure sensor (8), or variable according to variations of a dimension of the pressure sensor (8) caused by pressure variations, and capable of being detected, and the pressure sensor (8, 30) being in the form of a capacitor having a deformable membrane (9), spatially separated from the coil (13), and connected thereto by an electric conductor (15, 31, 35).

2. Device according to claim 1, characterized in that the pressure sensor (8, 30) is so designed that it is capable of being introduced in the medulla (4) of a bone (5) or attached to a bone (36).

3. Device according to claim 1, characterized in that the pressure sensor (8, 30) is so designed that it is capable of being fastened to or integrated in a prosthesis (6).

4. Device according to claim 1, characterized in that the pressure sensor (8, 30) is so designed that it is capable of being attached to the dentition.

5. Device according to any one of claims 1 to 4, characterized in that the pressure sensor (30) comprises an housing and the coil (13) and the electric circuit (14) are disposed in another housing (32, 34) and connected to each other by an electric conductor (31, 5) to form the probe.

6. Device according to any one of claims 1 to 4, characterized in that the pressure sensor (8), the coil (13), and an electric circuit (14) are disposed in a T-shaped housing (37), the pressure sensor (8) being disposed in the stem (11) and the coil (13) as well as the electric circuit (14) being disposed in the crosspiece and connected to each other by an electric conductor (15) to form the probe (1, 33).

7. Device according to claim 5 or 6, characterized in that the housing (32, 34; 37) containing the coil (13) and the electric circuit (14), and possibly the pressure sensor (8), is provided with bores (28) and screws (29) in order to be fastened to the cortex (21) of a bone (5, 36).

8. Device for the in vivo measurement of pressures and pressure variations inside or outside bones, comprising an implantable probe and an evaluating unit, characterized in that the probe (16) comprises an housing containing a capacitor (19) and a coil (22) which form an oscillating circuit (L-C), and the evaluating unit (3) includes a resonance oscillating circuit (7) capable of detecting the natural frequency (Fₑ) of the oscillating circuit, the natural frequency (Fₑ) of the oscillating circuit (L-C) being determined by the momentary pressure acting on the capacitor (19), or variable according to variations of a dimension of the capacitor (19) caused by pressure variations, and capable of being detected, and the housing of the capacitor including thinner portions (25) in order to be compressible.

9. Device according to claim 8, characterized in that the probe includes a capacitor (19) having compressible capacitor plates (20), the two outermost capacitor plates (23, 24) being fastened to the inside of the sides of the housing, which are displaceable relative to each other as well.

10. Device according to claim 8 or 9, characterized in that the probe (16) comprises a threaded pin (17, 18) which is designed to be screwed into the cortex (21) of a bone (5).

11. Device according to any one of claims 1 to 10, characterized in that the device includes an interface (2) which is designed to receive the signals of the probe and to transmit them to the evaluating device, the interface comprising a power supply.

12. Device according to claim 11, characterized in that the interface (2) comprises a long-life battery and is designed for implantation.

13. Device according to claim 11 or 12, characterized in that the interface (2) is composed of at least two portions which are connected to each other by a cable, one of the portions containing the antenna and the other portion containing the remaining electronics and the battery.
